(19) United States
(12) Patent Application Publication
(10) Pub. No.: US 2009/0023668 A1
(43) Pub. Date: Jan. 22, 2009

(54) Method for treating blepharitis

(76) Inventor: Mitchell H. Friedlaender, La Jolla, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/979,354

(22) Filed: Nov. 1, 2007

Related U.S. Application Data
(60) Provisional application No. 60/856,009, filed on Nov. 2, 2006.

(51) Int. Cl. A61K 31/573; A61P 29/00; A61K 31/7036

(52) U.S. Cl. 514/40; 514/171

(57) ABSTRACT

The present invention is related to a method for treating blepharitis, the method comprising administering to an ocular area of a subject in need thereof a composition comprising about 0.001% to about 0.01% (w/v) dexamethasone and an antibiotic, wherein the composition is substantially free of lipids, and wherein the composition is ophthalmically acceptable.

This application claims the benefit of the filing date of U.S. application Ser. No. 60/856,009, filed Nov. 2, 2006, the entirety of which is fully incorporated by reference herein.

### BACKGROUND OF THE INVENTION

1. Field of the Invention

The present invention is related to a method for treating blepharitis, the method comprising administering to an ocular area of a subject in need thereof a composition comprising about 0.001% to about 0.01% (w/v) dexamethasone and an antibiotic, wherein the composition is substantially free of lipids, and wherein the composition is ophthalmically acceptable.

2. Background Art

Blepharitis is a common ophthalmic condition, characterized by inflammation of the eyelids. Inflammation can be induced by irritants that collect on the eyelids and lashes, including oil, dandruff, mucin, bacteria, and environmental debris, such as pollutants, pollens, and eye makeup. Some, or all, of these irritants can form deposits on the eyelid margins. Bacteria, e.g., staphylococcus, thrive in this material, and can produce toxic products that contribute to the irritation of the surface of the eye.

Blepharitis is a common cause of “red eyes” and is especially common in people with fair complexions, particularly those with Scottish, Irish, or English backgrounds. The most prominent symptoms are burning, stinging, redness, and stickiness of the eyelids. Symptoms are generally most severe upon awakening because material, which accumulates on the eyelids at night, is not blinked away as it would be during waking hours.

Traditional treatments of blepharitis consist of removing debris from the eyelids and eye lashes, and/or treating the eyelids with antibiotics and anti-inflammatory medication. Various eye-lid cleansers have been developed to remove debris from the eyelids and eye lashes. Other methods of removing debris include the use of baby shampoo and water, applied with a wash cloth, in the shower, or at the sink.

Various medical treatments can be useful adjuncts to lid scrubs. Antibiotics can reduce bacterial populations on the eyelids. Anti-inflammatory medications, such as corticosteroids, can reduce inflammation. Antibiotics and corticosteroids have previously been used in petrolatum-based ointments, and have been administered separately or in combination with each other. Petroleum-based ointments, however, produce a film when they get inside the eye lids, producing blurred vision. While blurred vision from ointment can be tolerated while asleep, when awake, the ointment film can interfere with such activities as driving and reading.

Additionally, corticosteroids have been associated with ocular complications, such as cataracts, glaucoma, and opportunistic infections. To avoid these complications, medical professionals often avoid treatments with corticosteroids or antibiotic/corticosteroid combinations.

A need exists in the art for an antibiotic/corticosteroid composition for the treatment of blepharitis, which avoids an undesirable ointment film, and also avoids the complications of corticosteroids.

### BRIEF SUMMARY OF THE INVENTION

The present invention is related to a method for treating blepharitis, the method comprising administering to an ocular area of a subject in need thereof a composition comprising about 0.001% to about 0.01% (w/v) dexamethasone and an antibiotic, wherein the composition is substantially free of lipids, and wherein the composition is ophthalmically acceptable.

In some embodiments, the composition further comprises a carrier. In some embodiments, the carrier is selected from the group consisting of polyethylene glycol, polyvinyl alcohol, gelatin, hyaluronic acid, carbomer, tragacanth, a water-soluble cellulose derivative, colloidal magnesium aluminum silicate, sodium alginate, and combinations thereof. In some embodiments, the water-soluble cellulose derivative is selected from the group consisting of carboxymethyl cellulose, methyl cellulose, hydroxypropylmethyl cellulose, hydroxyethyl cellulose, and combinations thereof. In some embodiments, the soluble cellulose derivative is carboxymethyl cellulose.

In some embodiments, the carrier is about 0.1% to about 10.0% (w/v) of the composition. In some embodiments, the carrier is about 0.5% to about 2.0% (w/v) of the composition.

In some embodiments, the antibiotic is gentamicin or tobramycin. In some embodiments, the antibiotic is about 0.005% to about 0.5% (w/v) of the composition. In some embodiments, the antibiotic is about 0.01% to about 0.05% (w/v) of the composition.

In some embodiments, the dexamethasone is about 0.005% (w/v) of the composition.

In some embodiments, the composition is administered in a liquid state.

In some embodiments, administering the composition of the present invention does not result in an increase in intraocular pressure of the subject.

In some embodiments, the composition further comprises a tonicity agent, a pH adjuster, a preservative, a demulcent, a buffering agent, a lubricant, or combinations thereof. In some embodiments, the tonicity agent is selected from the group consisting of sodium chloride, calcium chloride, potassium chloride, magnesium chloride, boric acid, and combinations thereof.

In some embodiments, the pH adjuster is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof.

In some embodiments, the preservative is selected from the group consisting of benzalkonium chloride, ethylenediaminetetraacetic acid or salts thereof, Purite®, chlorobutanol, sodium perborate, sorbic acid, and combinations thereof.

In some embodiments, the demulcent is selected from the group consisting of a water-soluble cellulose derivative, dextran, gelatin, polyol, polyvinyl alcohol, povidone, chondroitin sulfate, hyaluronic acid, and combinations thereof.

In some embodiments, the buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

In some embodiments, the lubricant is selected from the group consisting of dextran, hydroxypropyl methylcellulose (HPMC), glycerin, polyethylene glycol (PEG) and propylene glycol.

In some embodiments, the dexamethasone is dexamethasone sodium phosphate.

In some embodiments, the composition comprises: (a) about 0.0005% to about 0.01% (w/v) dexamethasone; (b) about 0.005% to about 0.5% (w/v) gentamicin; and (c) about 0.1% to about 1.0% (w/v) carboxymethylcellulose.

In some embodiments, the composition comprises: (a) about 0.0005% to about 0.01% (w/v) dexamethasone; (b) about 0.005% to about 0.5% (w/v) gentamicin; and (c) about 0.1% to about 1.0% (w/v) carboxymethylcellulose, wherein the composition is substantially free of lipids.

In some embodiments, the composition comprises: (a) about 0.005% (w/v) dexamethasone; (b) about 0.02% (w/v) gentamicin; (c) about 0.625% (w/v) carboxymethylcellulose; and (d) about 0.015% (w/v) benzalkonium chloride; wherein the composition has a pH of about 4.0 to about 7.0, and wherein the composition is substantially free of lipids.

In some embodiments, the invention is directed to a kit comprising: (a) a composition comprising about 0.001% to about 0.01% (w/v) dexamethasone and an antibiotic, wherein the composition is substantially free of lipids, and wherein the composition is ophthalmically acceptable; and (b) instructions for using the composition of (a) for the treatment of blepharitis. In some embodiments, the kit further comprises a means for administering the composition. In some embodiments, the composition is individually packaged for a single administration.

### DETAILED DESCRIPTION OF THE INVENTION

The current invention relates to a method for treating blepharitis by administering a composition comprising an antibiotic and dexamethasone. The present invention is also related to a method for treating blepharitis, the method comprising administering to an ocular area of a subject in need thereof a composition comprising about 0.001% to about 0.01% (w/v) dexamethasone and an antibiotic, wherein the composition is substantially free of lipids, and wherein the composition is ophthalmically acceptable.

It is to be noted that the term “a” or “an” refers to one or more of that entity; for example, “a tonicity agent,” is understood to represent one or more tonicity agents. As such, the terms “a” (or “an”), “one or more,” and “at least one” can be used interchangeably herein. As used herein, “about” refers to plus or minus 10% of the indicated number. For example, “about 0.5%” indicates a range of 0.45% to 0.55%.

The compositions of the present inventions are “ophthalmically acceptable.” The term “ophthalmically acceptable” refers to those compounds, materials, compositions, and/or solutions which are, within the scope of sound medical judgment, suitable specifically for contact with the tissues of the eye, and the area surrounding the eye without excessive toxicity, irritation, allergic response, or other complications commensurate with a reasonable benefit/risk ratio.

Blepharitis, as used herein, refers to long-lasting or chronic inflammation of the eyelids, particularly at the lid margins. Symptoms associated with blepharitis include general eye discomfort, redness of the eye, excessive tearing, burning, stinging, foreign body sensation, itching, light sensitivity (photophobia), and/or an irritating, sandy, gritty sensation that is often worse upon awakening. Additional symptoms include red and swollen eyelids, blurred vision, frothy tears, and/or crusting of the eyelashes upon awakening. Severe and/or chronic effects of blepharitis can include thickened lid margins, dilated and visible capillaries, trichiasis, eyelash loss, ectropion and entropion. As used herein, treatment of blepharitis by the composition described herein can treat one, or more than one (e.g., two, three, four, five), of the above listed symptoms and effects.

In some instances, blepharitis can be classified as two different forms: anterior blepharitis and posterior blepharitis. Anterior blepharitis affects the outer margin of the eyelids, where the eyelashes are located. Posterior blepharitis affects the inner eyelid where the meibomian oil glands are located. Blepharitis may be anterior, posterior, or a combination of anterior and posterior. The method of the present invention can be used to treat one or both forms of blepharitis.

The present invention is directed to a method of treating blepharitis with a composition comprising an antibiotic and dexamethasone. Various ophthalmically acceptable antibiotics can be used in the present invention. Examples include, but are not limited to broad spectrum aminoglycoside antibiotics; e.g., gentamicin, kanamycin, neomycin, netilmicin, streptomycin, and tobramycin. Other classes of antibiotics within the scope of the present invention include, but are not limited to, tetracyclines, sulfonamides, quinolones, polypeptides (e.g., polymyxin B, colistin, bacitracin), penicillins, monobactam, macrolides, glycopeptides, cephalosporins (first, second, third, or fourth generation), carbapenems, and carbacephems. In some embodiments, the antibiotic is gentamicin or tobramycin. In some embodiments, the antibiotic is a broad spectrum antibiotic. In other embodiments, the antibiotic is a narrow spectrum antibiotic. In some embodiments, the antibiotic has high activity against gram positive bacteria; e.g., staphylococcus.

Various concentrations of antibiotic can be used in the present invention. In some embodiments, the antibiotic is about 0.001% to about 1.0% (w/v) of the composition, or about 0.005% to about 0.5% (w/v) of the composition. In some embodiments, the antibiotic is about 0.01% to about 0.05% (w/v) of the composition.

The term “dexamethasone,” also known as dexamethazone, refers to a corticosteroid of Formula I, or derivatives, salts or esters thereof.

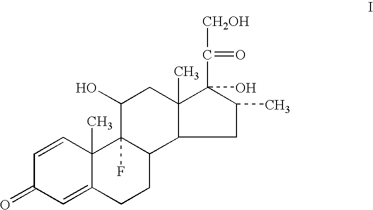

Thus, the term dexamethasone includes, but is not limited to; e.g., dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

Traditionally, dexamethasone has been administered to an ocular area at a concentration of 0.1%. However, numerous studies have shown that administration of dexamethasone at 0.1% can result in an increase in intraocular pressure. See e.g., Tripathi, R. C. et al., Drugs and Aging, 15:439-50 (1999). The present invention provides a method of administering dexamethasone to a subject at a concentration that does not result in an increase in intraocular pressure, yet still provides effective treatment for blepharitis.

In the present invention, dexamethasone is used in low concentrations, unlikely to produce any ocular complications, e.g. increase intraocular pressure. See, e.g. Shulman, D. G. et al., Opthamol. 106:362-9(1999). The present invention, has found that even at these low concentrations, dexamethasone can reduce the inflammation associated with blepharitis. The present invention can encompass various concentrations of dexamethasone. In some embodiments, the composition comprises about 0.0005% to about 0.05% (w/v), 0.001% to about 0.02% (w/v), or about 0.005% to about 0.01% (w/v) dexamethasone in the composition. In some embodiments, the composition comprises about 0.0025% to about 0.005% (w/v) dexamethasone in the composition. In some embodiments, the dexamethasone is about 0.005% (w/v) of the composition. One of skill in the art will recognize that composition of the present invention comprising dexamethasone can be manufactured, sold, stored or dispersed in concentrations that exceed 0.05%, and are later diluted to the ranges that fall within the scope of this invention.

The term “intraocular pressure” refers to the fluid pressure inside the eye. Intraocular pressure can be measured in various ways known to those in the art; e.g., intraocular pressure can be measured with a tonometer. Normal eye pressure, as measured by an eye doctor, usually ranges between 10 and 21 mm of mercury, with an average of 16. Thus, in some embodiments, the present invention is directed to a method of treating blepharitis with a composition comprising dexamethasone, and an antibiotic, wherein after administration of the composition the intraocular pressure of the subject being treated remains between 10 and 21 mm of mercury as measured by a tonometer. Intraocular pressure that is consistently above 21 indicates ocular hypertension. Ocular hypertension can lead to glaucoma, a serious disease that causes damage to the optic nerve. Thus, in some embodiments of the present invention is provided a method of treating blepharitis with a composition comprising dexamethasone, wherein there is not an increased risk of ocular hypertension or glaucoma.

In the present invention, the composition can be substantially free of lipids. The term “lipid” refers to hydrocarbon-based molecules of biological origin that are predominantly nonpolar or hydrophobic. The basic classes of lipids are: fatty acids (e.g., saturated or unsaturated fatty acids), glycerides or glycerolipids (e.g., monoglycerides, diglycerides, triglycerides (neutral fats), phosphoglycerides or glycerophospholipids), nonglycerides (e.g. sphingolipids, sterol lipids (includes cholesterol and steroid hormones)), prenol lipids (includes terpenoids), waxes, polyketides, and complex lipid derivatives (e.g., glycolipids, and protein-linked lipids). In some embodiments, the term lipid refers to petrolatum or petroleum. The term “petrolatum” refers to a substance which is a complex combination of semi-solid, saturated hydrocarbons, mainly of a paraffinic nature, obtained from petroleum. Generally, petrolatum comprises liquid hydrocarbons having carbon numbers predominately greater than C₂₅. Compositions substantially free of lipids can be beneficial when administering the composition of the present invention to an ocular area to avoid blurred vision due to an ointment film.

In some embodiments, the term “substantially free of lipids” refers to a composition wherein about 0% to 2% (w/w) of the composition is a lipid, about 0% to 1% (w/w) of the composition is a lipid, about 0% to 0.5% (w/w) of the composition is a lipid, or about 0% to 0.2% (w/w) of the composition is a lipid, or about 0% to 0.1% (w/w) of the composition is a lipid. In some embodiments, the term “substantially free of lipids” refers to compositions wherein less than 0.1% (w/w) of the composition is a lipid.

The present compositions can be in any physical state suitable to be administered to the eye such as, but not limited to, liquids (e.g., solutions or suspensions), semi-solids (gels, creams, ointments, etc.), and the like. Each of these physical states of the present compositions can be prepared using techniques and processes which are conventional and well known in the art. For a more detailed discussion of the preparation and administration of ophthalmic formulations see Remington's Pharmaceutical Sciences, 15 Ed., pgs. 1489 to 1504 (1975) which is incorporated in its entirety herein by reference. In some embodiments, the composition is administered in a liquid state.

The term “ocular area” refers to the external skin surrounding the eye; i.e., the eyelid and the margin of the eyelid, and associated hair projecting therefrom; i.e., eyelashes and eye brows. In some embodiments, the term “ocular area” also refers to the eyeball surface and the interior of the eyelid. The ocular surface can be present on any animal having an eyelid. Thus, methods of the present invention are applicable to both human use and veterinary use, preferably for human use. In some embodiments, the methods are applicable for use on domesticated animals such as companion animals (dogs and/or cats), livestock, or other economically important animals (e.g., model or breeding animals).

In some embodiments, the term “administering to an ocular area” includes placing the composition of the invention in direct contact with the ocular area, e.g., placing eye-drops or ointments containing the composition onto the ocular area, or by placing the composition onto an applicator, then contacting the applicator to the ocular area. In some embodiments, the term “administering” further includes agitating or rubbing the composition into the ocular area.

The terms “treating” or “treatment” refer to both therapeutic treatment and prophylactic or preventive measures, wherein the object is to prevent, inhibit, reverse or slow down (lessen) any undesired effect of blepharitis. For purposes of this invention, beneficial or desired results include, but are not limited to, alleviation of symptoms; diminishment of extent of the blepharitis, stabilized (i.e., not worsening) state of blepharitis, delay in onset of blepharitis, or slowing of progression of blepharitis; amelioration of blepharitis, remission of blepharitis (whether partial or total); or enhancement or improvement of the symptoms associated with blepharitis.

The method of the present invention can comprise administering the composition once daily or more than once daily (e.g., twice daily or three times per day). In some embodiments, the present invention can be administered on an “as needed” basis or on an “as desired” basis. In some embodiments, it is suitable to administer the composition once daily or twice daily to achieve relief from the symptoms of blepharitis. Single or twice daily administration can be beneficial for various reasons; e.g., single and twice daily administration can result in greater convenience and higher patient compliance.

Various amounts of the composition can be administered to an ocular surface. One of skill in the art will understand that the amount to be administered is dependent on various factors; e.g., the location of administration, the concentration of the dexamethasone, the concentration of the antibiotic, the viscosity of the composition, the frequency of administration, severity of the blepharitis, etc. In some embodiments, the amount of the composition to be administered is less than about 10 mL, about 0.01 mL to about 5 mL, about 0.1 mL to about 3 mL, or about 0.2 mL to about 1 mL. In some embodiments, the amount of the composition to be administered can be; e.g., 1 to 20 drops, 1 to 10 drops, or 2 to 5 drops.

In some embodiments, the composition further comprises a carrier. A carrier is any substance which is ophthamically suitable which by itself, generally has little or no therapeutic value and which does not react chemically with the dexamethasone, antibiotic, and/or any other constituents of the composition which may be present. In some embodiments, a carrier provides mass or volume to the dexamethasone and/or antibiotic to aid in application of the composition to a subject in need thereof. In some embodiments, the carrier is water soluble. The use of a water soluble carrier, rather than a water insoluble carrier, aids in the rapid dispersion of the product onto the ocular surface. Water soluble carriers also provide a benefit if the composition comes in contact directly with an eye, since it is more easily dissolved and dispersed on the eye and in the tears of the eye. These carriers provide increased comfort to a patient, and make the administration of the composition suitable for night time use, as well as day time use, since the composition does not blur the vision of the subject as much as a composition comprising a water insoluble carrier (e.g., a petrolatum-based composition). In some embodiments, the carrier is selected from the group consisting of polyethylene glycol, polyvinyl alcohol, gelatin, hyaluronic acid, carbomer, tragacanth, a soluble cellulose derivative, colloidal magnesium aluminum silicate, sodium alginate, and combinations thereof. In some embodiments, the water soluble cellulose derivative is selected from the group consisting of carboxymethyl cellulose (i.e., gum cellulose or CMC), methyl cellulose, hydroxypropylmethyl cellulose, hydroxyethyl cellulose, and combinations thereof. In some embodiments, the soluble cellulose derivative is carboxymethyl cellulose.

The term “water soluble” refers the ability of the composition to dissolve (i.e., form a solution) in water. One of skill in the art will recognize that solubility will be dependent on the volume of the solvent (i.e., water), the presence or absence of other compounds (e.g., solubilizing agents), the temperature of the solvent, as well as other factors. In some embodiments, the term “water soluble” refers to the ability of at least 90% of a substance to dissolve completely in water at room temperature in 1 hour, wherein the substance is 10% the volume of the water.

The composition can comprise various concentrations of the carrier. In some embodiments, the carrier is about 0.1% to about 10.0% (w/v) of the composition. In some embodiments, the carrier is about 0.5% to about 2.0% (w/v) of the composition. In some embodiments, the carrier is about 0.625% (w/v) of the composition.

In some embodiments, the term “substantially free of lipids” refers to a composition wherein about 0% to 2% (w/w) of the composition is a lipid, about 0% to 1% (w/w) of the composition is a lipid, about 0% to 0.5% (w/w) of the composition is a lipid, or about 0% to 0.2% (w/w) of the composition is a lipid, or about 0% to 0.1% (w/w) of the composition is a lipid. In some embodiments, the term “substantially free of lipids” refers to compositions wherein less than 0.1% of the composition is a lipid.

In some embodiments, the composition further comprises a tonicity agent, a pH adjuster, a preservative, a demulcent, a buffering agent, a lubricant, or combinations thereof.

Various tonicity agents can be used to adjust the salt concentration of the composition, provided the agent is ophthalmically acceptable. In some embodiments, the tonicity agent is selected from the group consisting of dextrose, sodium chloride, calcium chloride, potassium chloride, magnesium chloride, boric acid. and combinations thereof. In some embodiments, the tonicity agent is used to produce an isotonic composition. In some embodiments, the tonicity agent is used to produce a hypotonic composition.

Various pH adjusting agents can be used in the present invention. pH adjusting agents include any composition or compound capable of adjusting the pH of the composition to the desired level. pH adjusting agents can include acids and/or bases. In some embodiments, the pH adjusting agent is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof. One of skill in the art can determine the appropriate concentrations and amounts of the pH adjusting agent suitable for use when applying to an ocular area.

In some embodiments, the pH adjusting agent can be included to provide and/or maintain the composition of the present invention at a pH in a physiologically acceptable range; e.g., in a pH range of about 3 to about 9, about 4 to about 8.5, about 5 to about 8.5, or about 5.0 to about 7.0. In some embodiments, the pH adjusting agent provides and/or maintains a pH of about 4.0 to about 7.0 or about 5.0 to about 6.0. As one of skill in the art will recognize, the pH can vary over time, depending on various factors; e.g., stability of the composition, amount of exposure to the atmosphere, etc. Thus, as used herein, when referring to compositions, kits or methods of the present invention, any specified pH refers to the pH at any time between the time of manufacturing and time of administering. In some embodiments, the composition remains in a slightly acidic pH, since dexamethasone is more stable in a slightly acidic pH.

Acids useful as pH adjusting agents in the present compositions can include boric acid, hydrochloric acid, acetic acid, organic acids, other acids which are ophthalmically acceptable in the concentrations and pH levels used, and the like.

Bases useful as pH adjusting agents in the present compositions include, but are not limited to, sodium and/or potassium hydroxides, other alkali and/or alkaline earth metal hydroxides, organic bases, other bases which are ophthalmically acceptable in the concentrations and pH levels used, and the like.

In some embodiments of the present invention the composition comprises a preservative. The term “preservative” refers to any substance that can keep the composition chemically stable, and/or can inhibit the growth of microorganisms in the composition. For example, a preservative can protect the dexamethasone from instability caused by light, moisture, heat, or oxidation. In some embodiments, the preservative can be an antioxidant. In some embodiments, the preservative can be an antimicrobial. Preservatives include, but are not limited to, α-lipoic acid, α-tocopherol, ascorbyl palmitate, benzyl alcohol, biotin, bisulfites, butylated hydroxyanisole (BHA), butylated hydroxytoluene (BHT), ascorbic acid and its esters, carotenoids, calcium citrate, acetyl-L-carnitine, chelating agents, chondroitin, citric acid, coenzyme Q-10, cysteine, cysteine hydrochloride, 3-dehydroshikimic acid (DHS), EDTA (ethylenediaminetetraacetic acid) and salts thereof, ferrous sulfate, folic acid, fumaric acid, niacin, tocopherol and their esters; e.g., tocopherol acetate, tocopherol succinate, tocotrienal, d-α-tocopherol acetate, vitamin A and its esters, vitamin B and its esters, vitamin C and its esters, vitamin D and its esters, vitamin E and its esters; e.g., vitamin E acetate, and combinations thereof. In some embodiments, the preservative is selected from the group consisting of benzalkonium chloride, EDTA, Purite® (Allergan, Irvine, Calif.), chlorobutanol, sodium perborate, sorbic acid, and combinations thereof.

Various concentrations of preservative can be used in the composition of the invention, dependant on; e.g., the type of preservative, its mechanism of action, and/or its ophthalmic acceptability. In some embodiments, the preservative is about 0.00001% to about 4.0% (w/v) of the composition, or about 0.0001% to about 1.0% (w/v) of the composition, or about 0.001% to about 0.1% (w/v) of the composition.

Various demulcents can be used. The term “demulcent” refers to any compound or composition that when applied to an ocular area can lubricate, soothe and/or protect the mucous membrane of the eye. In some embodiments, the demulcent is selected from a water soluble cellulose derivative, dextran, gelatin, polyol, polyvinyl alcohol, povidone, chondroitin sulfate, hyaluronic acid, and combinations thereof. Various water soluble cellulose derivatives can be used. Examples include, but are not limited to, carboxymethylcellulose, one or more salts of carboxymethylcellulose, hydroxyethyl cellulose, hypromellose, methylcellulose, and combinations thereof. The term “polyol” refers to a compound with greater than two alcohol groups. Examples of polyols include, but are not limited to glycerin, polyethylene glycol, polysorbate, propylene glycol, and combinations thereof. Various concentrations of demulcents can be used in the present invention. In some embodiments, the demulcent is about 0.01% to about 20.0% (w/v) of the composition, about 0.1% to about 10.0% (w/v) of the composition, or about 0.5% to about 5.0% (w/v) of the composition.

In some embodiments, the composition comprises a buffering agent. The terms “buffer” or “buffering agent” refer to an ophthalmically acceptable compound or composition that can neutralize both acids and bases, thereby maintaining the original acidity or basicity of the composition. Buffers can include, but are not limited to, phosphate buffers (e.g., sodium and potassium phosphates), phosphates, bicarbonate, citrate, borate, acetate buffers, citrate buffers, tromethamine buffers and combinations thereof. Preferred buffers are selected from the group consisting of citric acid, sodium citrate, boric acid, sodium borate, one or more sodium salts of phosphoric acid, one or more potassium salts of phosphoric acid, sodium bicarbonate, and combinations thereof.

In some embodiments, the composition of the invention further comprises a lubricant. In some embodiments, the lubricant is selected from the group consisting of dextran, hydroxypropyl methylcellulose (HPMC), glycerin, polyethylene glycol (PEG) and propylene glycol.

In other embodiments of this invention, water, or water-based solvents (e.g., Ringers solution), can be added to the composition to provide the desired consistency of the composition. Various amounts of water or water-based solvent can be used. In some embodiments, the composition can comprise about 5% to about 50% (v/v), about 10% to about 40% (v/v), or about 15% to about 30% (v/v) water or water-based solvent. In some embodiments, the compositions can comprise about 50% to 99.9% (v/v), about 70% to 99% (v/v), or about 80% to about 97% (v/v) water or water-based solvent. In some embodiments, the compositions can comprise about 98%, about 98.5%, about 99%, about 99.5%, about 99.8% or about 99.9% water or water-based solvent.

The composition can comprise various amounts of the corticosteroid, e.g., dexamethasone, antibiotic, and carrier. In some embodiments, the composition comprises: (a) about 0.0005% to about 0.01% (w/v) dexamethasone; (b) about 0.005% to about 0.5% (w/v) gentamicin; and (c) about 0.1% to about 1.0% (w/v) carboxymethylcellulose. In some embodiments, the composition comprises: (a) about 0.0005% to about 0.01% (w/v) dexamethasone; (b) about 0.005% to about 0.5% (w/v) gentamicin; and (c) about 0.1% to about 1.0% (w/v) carboxymethylcellulose, wherein the composition is substantially free of lipids. In some embodiments, the composition comprises: (a) about 0.005% (w/v) dexamethasone; (b) about 0.02% (w/v) gentamicin; (c) about 0.625% (w/v) carboxymethylcellulose; and (d) about 0.015% (w/v) benzalkonium chloride; wherein the composition has a pH of about 4.0 to about 7.0, and wherein the composition is substantially free of lipids.

In some embodiments, the invention is directed to a kit comprising: (a) a composition comprising about 0.001% to about 0.01% (w/v) dexamethasone and an antibiotic, wherein the composition is substantially free of lipids, and wherein the composition is ophthalmically acceptable; and (b) instructions for using the composition of (a) for the treatment of blepharitis.

In some embodiments, the kit further comprises a means for administering the composition. In some embodiments, the means for administering can include a bottle, dropper, cup, specialized eye-wash apparatus, wetted towel or sponge. In some embodiments, the kit comprises a cleaning apparatus (e.g., a towel, pad, cloth, brush, sponge, etc.) and/or a cleaning solution (e.g., purified water, a detergent solution, a boric acid solution, etc.). In some embodiments of the present invention, the ocular area is cleaned prior to administration of the composition of the present invention.

The composition can be individually packaged for a single administration; e.g., in a bottle, jar, ampoule, tube, syringe, envelope, container, or vial. When the composition is individually packaged, in some embodiments the composition does not comprise a preservative. Alternatively, the composition can be contained in a package that is capable of holding multiple units; e.g., in resealable glass or plastic packages. In some kits, the components of the composition are mixed together immediately preceding their usage. For example, in some embodiments one or more dry components of the composition of the kit are packaged in a separate container; e.g., a plastic bottle, and then mixed with one or more of the liquid components of the composition immediately prior to use. Optionally, the kit of the present invention can include a dropper or other device for transferring or administering the composition to a subject.

Optionally, the kit can further comprise printed matter containing instructions for using the composition of the present invention. For example, such printed instructions can be in a form prescribed by a governmental agency regulating the manufacture, use or sale of pharmaceuticals or biological products, which reflects approval by the agency of the manufacture, use or sale for human application. In some embodiments, the kit further comprises printed matter, which, e.g., provides information on the use of the composition or a pre-recorded media device which, e.g., provides information on the use of the method of the present invention.

“Printed matter” can be, for example, a book, booklet, brochure, leaflet or the like. The printed matter can describe the use of the composition of the present invention. Possible formats include, but are not limited to, a bullet point list, a list of frequently asked questions (FAQ) or a chart. Additionally, the information to be imparted can be illustrated in non-textual terms using pictures, graphics or other symbols.

The kit can also include a container for storing the components of the kit. The container can be, for example, a bag, box, envelope or any other container suitable for use in the present invention. In some embodiments, the container is large enough to accommodate each component of the present invention. However, in some cases, it can be desirable to have a smaller container which is large enough to carry only some of the components of the present invention.

### EXAMPLES

A composition comprising 0.005% dexamethasone/0.02% gentamicin sulfate/0.625% carboxymethyl cellulose was prepared as outlined in Table 1.

TABLE 1

| Ingredient | Percent w/v |
|---|---|
| Dexamethasone Sod Phosphate | 0.005 |
| Gentamicin Sulfate | 0.02 |
| Benzalkonium Chloride | 0.015 |
| Sodium Carboxymethylcellulose | 0.625 |
| Sodium Hydroxide | pH 6-7 |
| Ringers Solution | q.s. 100 |

### Example 2

10 subjects presenting with symptoms of blepharitis were treated with the composition of Example 1. Symptoms included: redness, burning, stinging, foreign body sensation, stickiness and crusting of the lids, and discharge. Patients were instructed to rub the medication into the eyelids, with eyes closed, twice daily for two weeks. At the end of two weeks, 4 subjects reported marked improvement in the relief of symptoms associated with blepharitis, 2 subjects had minimal or no improvement, and 4 subjects did not report whether they had any improvement. No adverse side effects were observed or reported.

It is to be appreciated that the Detailed Description section, and not the Summary and Abstract sections, is intended to be used to interpret the claims. These examples illustrate possible compositions used in the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method for treating blepharitis, the method comprising administering to an ocular area of a subject in need thereof a composition comprising about 0.001% to about 0.01% (w/v) dexamethasone and an antibiotic, wherein the composition is substantially free of lipids, and wherein the composition is ophthalmically acceptable.

2. The method of claim 1, wherein the composition further comprises a carrier.

3. The method of claim 2, wherein the carrier is selected from the group consisting of polyethylene glycol, polyvinyl alcohol, gelatin, hyaluronic acid, carbomer, tragacanth, a water soluble cellulose derivative, colloidal magnesium aluminum silicate, sodium alginate, and combinations thereof.

4. The method of claim 3, wherein the carrier is a soluble cellulose derivative and the water soluble cellulose derivative is selected from the group consisting of carboxymethyl cellulose, methyl cellulose, hydroxypropylmethyl cellulose, hydroxyethyl cellulose, and combinations thereof.

5. The method of claim 4, wherein the water soluble cellulose derivative is carboxymethyl cellulose.

6. The method of claim 2, wherein the carrier is about 0.1% to about 10.0% (w/v) of the composition.

7. The method of claim 6, wherein the carrier is about 0.5% to about 2.0% (w/v) of the composition.

8. The method of claim 1, wherein the antibiotic is gentamicin or tobramycin.

9. The method of claim 1, wherein the antibiotic is about 0.005% to about 0.5% (w/v) of the composition.

10. The method of claim 9, wherein the antibiotic is about 0.01% to about 0.05% (w/v) of the composition.

11. The method of claim 1, wherein the dexamethasone is about 0.005% (w/v) of the composition.

12. The method of claim 1, wherein said administering does not result in an increase in intraocular pressure of the subject.

13. The method of claim 1, wherein said composition further comprises a tonicity agent, a pH adjuster, a preservative, a demulcent, a buffering agent, a lubricant, or combinations thereof.

14. The method of claim 13, wherein said tonicity agent is selected from the group consisting of sodium chloride, calcium chloride, potassium chloride, magnesium chloride, boric acid, and combinations thereof.

15. The method of claim 13, wherein said pH adjuster is selected from the group consisting of hydrochloric acid, acetic acid, sodium hydroxide, potassium hydroxide, an alkali earth metal hydroxide, an alkaline earth metal hydroxide, an organic base, an organic acid, and combinations thereof.

16. The method of claim 13, wherein said preservative is selected from the group consisting of benzalkonium chloride, ethylenediaminetetraacetic acid or salts thereof, Purite®, chlorobutanol, sodium perborate, sorbic acid, and combinations thereof.

17. The method of claim 13, wherein said demulcent is selected from the group consisting of a water soluble cellulose derivative, dextran, gelatin, polyol, polyvinyl alcohol, povidone, chondroitin sulfate, hyaluronic acid, and combinations thereof.

18. The method of claim 13, wherein said buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

19. The method of claim 13, wherein said lubricant is selected from the group consisting of dextran, hydroxypropyl methylcellulose (HPMC), glycerin, polyethylene glycol (PEG) and propylene glycol.

20. The method of claim 1, wherein the dexamethasone is dexamethasone sodium phosphate.

21. The method of claim 2, wherein:
(a) the dexamethasone is about 0.0005% to about 0.01% (w/v) of the composition;
(b) the antibiotic is gentamicin in a concentration of about 0.005% to about 0.5% (w/v) of the composition; and
(c) the carrier is carboxymethylcellulose in a concentration of about 0.1% to about 1.0% (w/v) of the composition.

22. A composition comprising:
(a) about 0.0005% to about 0.01% (w/v) dexamethasone;
(b) about 0.005% to about 0.5% (w/v) gentamicin; and
(c) about 0.1% to about 1.0% (w/v) carboxymethylcellulose,
wherein the composition is substantially free of lipids.

23. A composition comprising:
(a) about 0.005% (w/v) dexamethasone;
(b) about 0.02% (w/v) gentamicin;
(c) about 0.625% (w/v) carboxymethylcellulose; and
(d) about 0.015% (w/v) benzalkonium chloride;
wherein the composition has a pH of about 4.0 to about 7.0, and wherein the composition is substantially free of lipids.

24. A kit comprising:
(a) a composition comprising about 0.001% to about 0.01% (w/v) dexamethasone and an antibiotic, wherein the composition is substantially free of lipids, and wherein the composition is ophthalmically acceptable; and
(b) instructions for using the composition of (a) for the treatment of blepharitis.

25. The kit of claim 25, further comprising a means for administering the composition.
